(19) United States
(12) Patent Application Publication
(10) Pub. No.: US 2024/0257425 A1
(43) Pub. Date: Aug. 1, 2024

(54) Software for characterising a two-dimensional image obtained by means of an examination through images

(71) Applicants: Paolo TINAZZI MARTINI, Verona (IT); Matilde TINAZZI MARTINI, Verona (IT)

(72) Inventors: Paolo TINAZZI MARTINI, Verona (IT); Matilde TINAZZI MARTINI, Verona (IT)

(21) Appl. No.: 18/565,614

(22) PCT Filed: May 13, 2022
(86) PCT No.: PCT/IB2022/054486

§ 371 Date: Nov. 30, 2023

(30) Foreign Application Priority Data
Jun. 1, 2021 (IT) 102021000014342

(51) Int. Cl. G06T 11/60; G16H 30/40

(52) U.S. Cl. CPC G06T 11/60; G16H 30/40

(57) ABSTRACT

A software is described for characterising a two-dimensional image (20) obtained by means of an examination through images, comprising a computer code (1) which, when executed by a computer (10), performs the following steps: the step (S1) of allowing the acquisition of the two-dimensional image (20), the step (S2) of identifying a contour (25,30,35,40,45,50,55,60,65,70,75,80) of at least one anatomical part illustrated in the two-dimensional image (20), the step (S3) of generating a two-dimensional image (90) of the contour (25,30,35,40,45,50, 55,60,65,70,75,80), the step (S4) of displaying said two-dimensional image (90) of the contour (25,30,35,40,45,50,55,60,65,70,75,80), the step (S5) of making available an archive (95) of medical characteristics, each of which is associated with a respective character string, which provides a description of the medical characteristic, and with a respective distinctive colour, the step (S6) of displaying said archive of medical characteristics, the step (S7) of allowing the selection of a medical characteristic, the step (S8) of allowing the association of the medical characteristic to the contour (25,30,35,40, 45,50,55,60,65,70,75,80) of the two-dimensional image (90), the step (S9) of filling the two-dimensional image (90) of the contour with the colour of the respective selected medical characteristic, filling with colour only the portion of said two-dimensional image that is within the contour (25,30,35,40,45, 50,55,60,65,70,75,80), and the step (S10) of generating a report (R) containing a two-dimensional image (100) in which the contour is filled with the colour of the previously selected medical characteristic and which contains the character string associated with the previously selected medical characteristic.

### TECHNICAL FIELD

The present invention relates to software for characterising a two-dimensional image obtained by means of an examination through images, in particular to a software for characterising a two-dimensional image obtained by means of an examination through images adapted to generate a standardised medical report.

### PRIOR ART

Medical examinations through images, such as ultrasound, computed axial tomography and magnetic resonance imaging, result in a series of greyscale images, each of which illustrating a section, e.g., axial, of the patient's body.

These images, see for example FIG. 1, are not immediately interpretable, as the resolution is not particularly high and in many cases the boundary between one tissue type and another is not well defined.

The reporting physicians are in charge of analysing the images and writing a report in which they explain in words the patient's condition that can be extrapolated from the study of the images, such as the conformation of the organs and the possible presence of tumour lesions.

A problem with this method of reporting is that a second physician, e.g., a surgeon, when he has to read the report must necessarily look up what the reporting doctor has said in the images of the medical examination, which as mentioned above are not easy to interpret.

A further problem with the current reporting methodology is that the accuracy of the report and the comprehensibility thereof by third parties depends on the skills of each individual reporting physician, so it may be inaccurate and/or incomprehensible to another physician reading the report.

An object of the present invention is to make available a reporting assistance software which allows third parties to interpret the report without necessarily having to analyse the images of the medical examination.

Such object is achieved by the features of the invention indicated in the independent claim.

The dependent claims outline preferred and/or particularly advantageous aspects of the invention.

### DISCLOSURE OF THE INVENTION

The invention makes available a software for characterising a two-dimensional image obtained by means of an examination through images, comprising a computer code which, when executed by an electronic processor, performs the following steps:
the step of allowing the acquisition of the two-dimensional image,
the step of identifying a contour of at least one anatomical part illustrated in the two-dimensional image,
the step of generating a two-dimensional image of the contour,
the step of displaying said two-dimensional image of the contour,
the step of making available an archive of medical characteristics, each of which is associated with a respective character string, which provides a description of the medical characteristic, and with a respective distinctive colour,
the step of displaying said archive of medical characteristics,
the step of allowing the selection of a medical characteristic,
the step of allowing the association of the medical characteristic to the contour of the two-dimensional image,
the step of filling the two-dimensional image of the contour with the colour of the respective selected medical characteristic, filling with colour only the portion of said two-dimensional image that is within the contour,
the step of generating a report containing the two-dimensional image of the contour previously filled with the colour and character string associated with the selected medical characteristic.

Thanks to this solution, the time for third parties to understand the report is improved compared to the usual reporting methodology, as for example a surgeon studying the report for a surgical operation does not have to interpret the images. It can also be said that thanks to the simplification of the contours and the specific colours, the report turns from being an image that can be interpreted by a few into an image that can be understood by all. Furthermore, the fact of starting from the contour of the real anatomical element allows the surgeon to be prepared for what he will actually be facing, simplifying the choice of the surgical operations to be carried out.

According to one aspect of the invention, the computer code may comprise, subsequent to the step of displaying said two-dimensional image of the contour, the step of making available a contour editing tool.

In this way, the user of the software can make corrections to the contour in order to make it more closely matching with the two-dimensional image of the medical examination in case the software has made an error or inaccuracy during the contour detection step.

According to another aspect of the invention, the steps may be such that:
in the step it is envisaged identifying a respective contour for each anatomical part illustrated in the two-dimensional image,
in the step it is envisaged generating a two-dimensional image containing the contours of the identified anatomical parts,
in the step it is envisaged allowing the selection of another medical characteristic,
in the step, it is envisaged allowing the association of the other medical characteristic to another contour of the plurality of contours present in the two-dimensional image of the contours of the anatomical parts,
in the step it is envisaged filling the two-dimensional image of the contours of the anatomical parts with the colour of the other respective selected medical characteristic, filling with the colour only the portion of said two-dimensional image which is within the other contour to which the other medical characteristic has been associated,
in the step it is envisaged that the report contains the two-dimensional image of the contours previously filled with the colour and the character strings associated with the selected medical characteristics.

This makes the software more versatile and efficient.

According to yet another aspect of the invention, the computer code may comprise, subsequent to the step of step of displaying said two-dimensional image of the contour, the step of making a contour deletion tool available.

In this way, in the event that the software has mistakenly detected non-existent contours, it is possible to eliminate them in order to make the report more faithful to reality. This technical characteristic also provides the possibility of eliminating contours at will in the event that one wishes to focus the attention only on certain anatomical parts and not all those detected by the software in the two-dimensional image.

### BRIEF DESCRIPTION OF THE DRAWINGS

Further characteristics and advantages of the invention will become clear from reading the following description provided by way of non-limiting example, with the aid of the figures illustrated in the accompanying tables.

FIG. 1 is an image of a medical examination through images.

FIG. 2 is a diagram of an electronic processor according to the invention.

FIG. 3 is a software-generated image according to the invention, which contains the contours of the anatomical elements present in the image of FIG. 1.

FIG. 4 is an elaboration of FIG. 3 obtained by means of the software according to the invention, wherein the image portions within the contours are coloured according to a predetermined colour associated with a medical characteristic which by means of the software has been associated with the contour.

FIG. 5 is a schematic representation of a report generated by the software.

FIG. 6 is a diagram of the software operation.

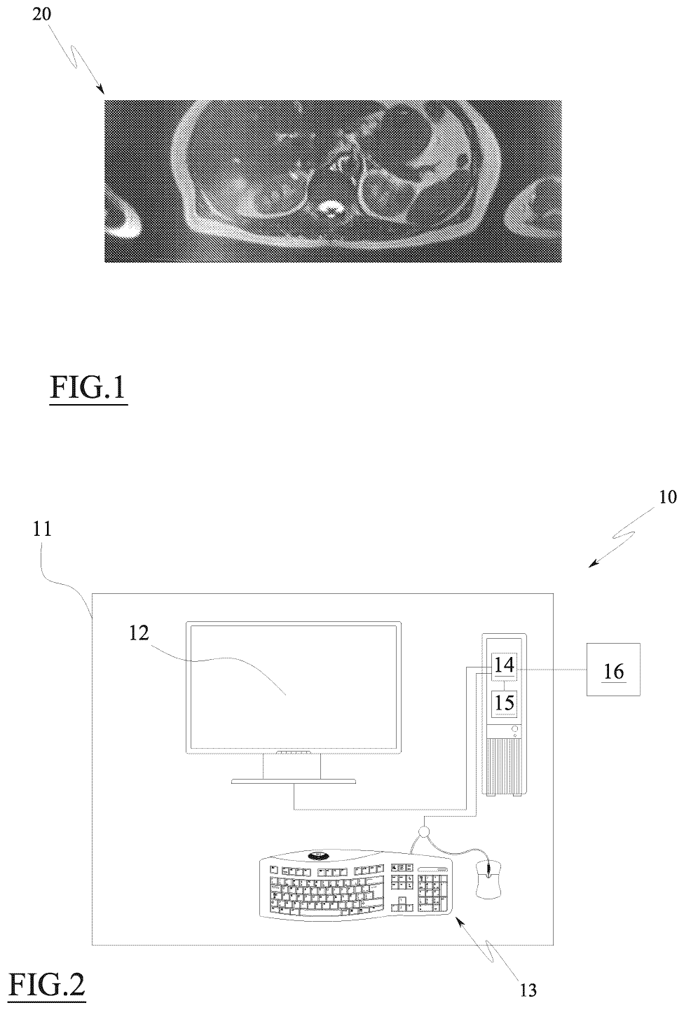

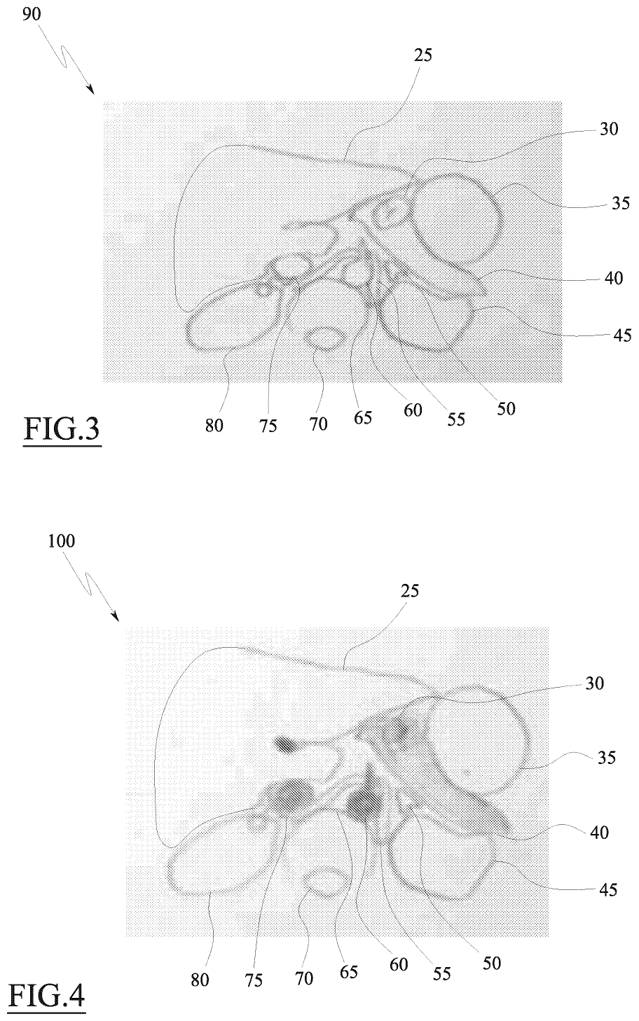

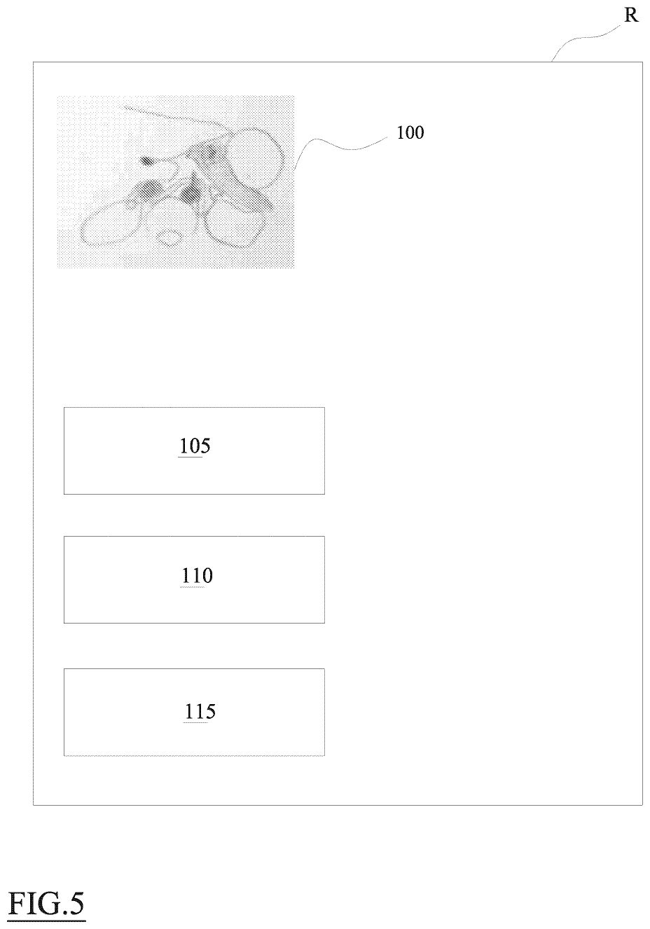

### BEST MODE OF THE INVENTION

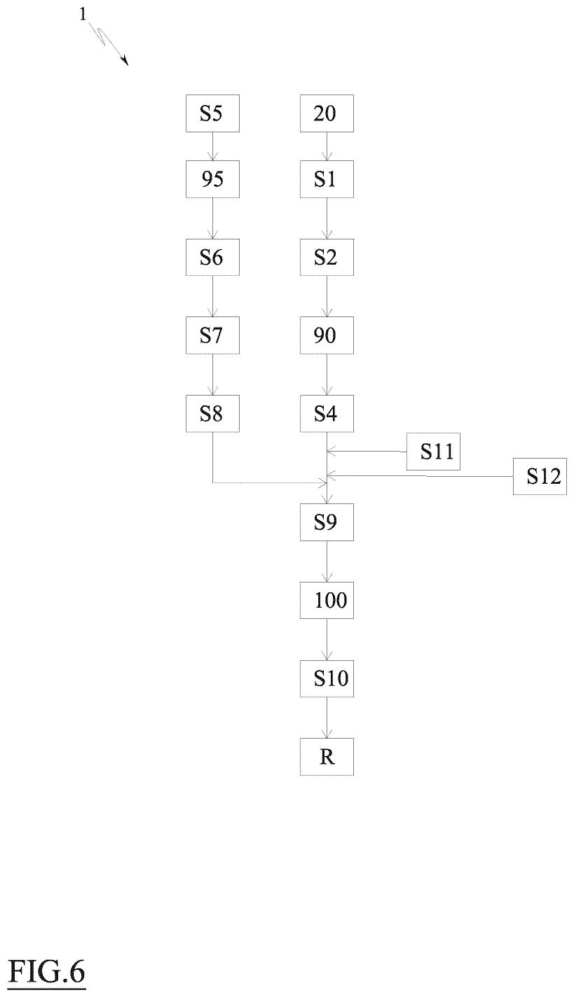

The numeral 10 is used to denote an electronic processor adapted to execute the computer code 1 (see diagram in FIG. 6) of a software for characterising a two-dimensional image obtained by means of an examination through images, such as an ultrasound, a computed axial tomography or a magnetic resonance imaging. Preferably the software is aimed at generating a report R for said medical examination.

The software is adapted to be used by a user who performs an analysis of the images of the medical examination, e.g., a reporting doctor.

The electronic processor 10 is, for example, a computer 11 on which the software is installed and through which the user gives commands to carry out the characterisation of the anatomical part.

Said computer comprises a display unit, for example a monitor 12, an input unit 13, a calculation unit 14, a volatile memory unit, and a permanent memory unit 15, for example a hard disk.

The input unit 13 comprises a keyboard, a mouse.

The input unit 13 may also comprise a microphone for voice input.

In a preferred embodiment, the electronic processor 10 comprises a further computer 16, which is connected via a network to the computer 11 and is provided with a permanent memory unit in which the computer code 1 of the software is stored.

It is not excluded that in another embodiment the computer code 1 may be stored in the permanent memory 15 of the computer 11.

The computer code 1, when executed by the electronic processor 10, performs a plurality of steps that allow the characterization of the two-dimensional image 20 in order to obtain said report R.

For the sake of brevity, it will be omitted in the remainder of the discussion that the step performed by the computer code 1 is executed when the computer code 1 is executed by electronic processor 10.

The computer code 1 performs step S1 of allowing the acquisition of the two-dimensional image 20 of the medical examination through images.

This step may envisages selecting the two-dimensional image 20 using a resource exploration tool of the processor and/or a drag-and-drop tool made available by the computer code.

This step may also envisages acquiring a plurality of two-dimensional images 20 belonging to a single medical examination through images.

Subsequent to step S1, the computer code performs step S2 of identifying a contour 25,30,35,40,45,50,55,60,65,70,75,80 (or in other words an edge) of at least one anatomical part illustrated in the two-dimensional image 20, i.e., in each acquired two-dimensional image 20.

It should be noted that contour means a closed line identifying the separation of one anatomical part from another anatomical part.

Preferably, step S2 envisages identifying a respective contour 25,30,35,40,45,50,55,60,65,70,75,80 (or in other words an edge) for each anatomical part illustrated in the two-dimensional image 20, i.e., in each acquired two-dimensional image 20.

This step of identifying the contour is known in the medical software industry as image segmentation. The algorithms used to perform the segmentation of a two-dimensional image obtained from a medical examination are varied and constantly evolving. Generally, such algorithms are based on the contrast between light and dark pixels of the two-dimensional image of the medical examination and/or other parameters, which often vary from one type of examination to another. Since they are known, they will not be described in detail. By way of example, algorithms of this type are described in EP2502546A1.

Subsequent to step S2, the computer code performs step S3 of generating a two-dimensional image 90 (digital) of the contour 25,30,35,40,45,50,55,60,65,70,75,80, i.e., of generating a two-dimensional image 90 containing the contour (the closed line indicative of the contour).

Preferably, step S3 generates a two-dimensional image 90 containing all the contours 25,30,35,40,45,50,55,60,65,70,75,80 of the anatomical elements identified in step S2.

Such an image may have a background with the contour in contrast with respect to the background, e.g., the background may be transparent or white and the contour may be represented in black.

“In contrast” means that the contour must be clearly visible to the human eye compared to the rest of the background. In other words, the pixels of the two-dimensional digital image 90 that form the contour, i.e., the contours, must be clearly distinguishable from the pixels that form the rest of the image.

The two-dimensional image 90 has an outer perimeter, i.e., an outer boundary, for example with the same shape as the one of the starting image 20, in the illustrated embodiment it is rectangular, where within said perimeter there is the contour, i.e., the contours.

The computer code then envisages step S4 of displaying said two-dimensional image 90 of the contour, i.e., the contours.

For example, this is displayed through monitor 12.

Immediately after step S4 or simultaneously or even before, the computer code performs step S5 of making available an archive 95 of medical characteristics, each of which is associated with a respective character string, which provides a description of the medical characteristic. Each medical characteristic is also associated with a distinctive colour that is different for each medical characteristic.

The colours and the strings are preset and unique for each medical characteristic, in order to achieve a standardised report, as will become clearer later on.

The colours can for example be: yellow, red, blue, green, light blue, pink, purple, etc.

For example, each medical characteristic is associated with a plurality of character strings, each of which composing, in a different language, the same description of the medical characteristic to which they refer.

The informed code then performs step S6 of displaying said archive of medical characteristics, in particular of displaying a symbol or string of characters indicative of the medical characteristic, for example through the monitor 12. For example, the archive can be displayed as a drop-down menu.

The computer code also performs step S7 of allowing the selection of a medical characteristic from the plurality of medical characteristics.

This can be done by means of the input unit 13, e.g., a mouse click.

The computer code then envisages performing step S8 of allowing the association of the selected medical characteristic to the contour of the two-dimensional image, i.e., a contour of the plurality of contours of the two-dimensional image.

This can be done, for example, by a drag-and-drop operation of the selected medical characteristic within or on the contour.

Steps S7 and S8 may be repeated so as to associate a medical characteristic with more than one contour identified by the medical examination image, e.g., so as to associate all contours with a medical characteristic.

The colours of the medical characteristics are in contrast with respect to the background of the two-dimensional image and to the contour, e.g., the background can be transparent or white, the contour can be represented in black and the colours can be yellow, red, blue, green, orange, pink, etc.

“In contrast” means that the contour must be clearly visible to the human eye compared to the rest of the background. In other words, the pixels of the two-dimensional digital image 90 that form the contour, i.e., the contours, must be clearly distinguishable from the pixels that form the rest of the image.

The computer code then performs, after step S8, i.e., after each step S8, step S9 of filling with the colour of the respective selected medical characteristic the portion of the two-dimensional image 90 that is within the contour to which the medical characteristic has been associated. Thus, a modified two-dimensional image 100 is obtained, in which the portion of the image within at least one contour is coloured with the colour associated with the medical characteristic selected for that contour.

Finally, the computer code comprises step S10 of generating the report R containing the two-dimensional image 100 of the contour previously filled with colour, i.e., the two-dimensional image 90 in which a plurality of portions within the respective contours has been filled with a respective colour of the medical characteristic associated in step S8, and the character string 105,110,115 associated with the selected medical characteristic, i.e., the plurality of medical strings corresponding to the associated medical characteristics.

The report R is therefore a two-dimensional image comprising at least the two-dimensional image 100 and the character strings associated with the selected medical characteristics, i.e., images illustrating said character strings.

The generation of the report can envisages generating the report R as a digital file which, when acquired by specific programs, allows it to be displayed on a monitor and/or printed on paper.

The computer code may also comprise other functions, such as for example allowing further character strings to be entered into the report using the keyboard of the processor. The computer code may also comprise, subsequent to step S4, step S11 of making available a tool for graphically editing the contour 25,30,35,40,45,50,55,60,65,70,75,80, i.e., one or a plurality of contours generated in step S3.

In particular, this tool for graphically editing the contour allows the position of one or more contour points to be shifted with respect to a fixed reference point on the two-dimensional image (e.g., the centre of the image or a corner thereof). This allows the shape of the contour to be corrected at one or more points.

In practice, this tool makes it possible to change the position of the pixels of the contour of the two-dimensional digital image with respect to a fixed reference pixel of the two-dimensional image.

The computer code may also comprise, subsequent to step S4, step S12 of making a tool of deletion of a contour available, i.e., of one or more contours of the plurality of contours generated in step S3, from the two-dimensional image 90.

This tool basically allows a contour or more contours to be selected and the computer code after this selection performs the step of removing the contour from the two-dimensional image.

In practice, this can be done by transforming the contour pixels into the background pixels of the two-dimensional digital image.

The invention thus conceived is susceptible to several modifications and variations, all falling within the scope of the inventive concept.

Moreover, all the details can be replaced by other technically equivalent elements.

In practice, the materials used, as well as the contingent shapes and sizes, can be whatever according to the requirements without for this reason departing from the scope of protection of the following claims.

1. Software for characterising a two-dimensional image obtained by an examination through images, comprising a computer code embodied on a non-transitory computer readable medium, which, when executed by an electronic processor, performs the following steps:
the step of allowing the acquisition of the two-dimensional image,
the step of identifying a contour of at least one anatomical part illustrated in the two-dimensional image,
the step of generating a two-dimensional image of the contour,
the step of displaying said two-dimensional image of the contour,
the step of making available an archive of medical characteristics, each of which is associated with a respective character string, which provides a description of the medical characteristic, and with a respective distinctive colour,
the step of displaying said archive of medical characteristics,
the step of allowing the selection of a medical characteristic,
the step of allowing the association of the medical characteristic to the contour of the two-dimensional image,
the step of filling the two-dimensional image of the contour with the colour of the respective selected medical characteristic, filling with colour only the portion of said two-dimensional image that is within the contour, and
the step of generating a report containing a two-dimensional image in which the contour is filled with the colour of the previously selected medical characteristic and which contains the character string associated with the previously selected medical characteristic.

2. Software according to claim 1, comprising, subsequent to the step of displaying said two-dimensional image of the contour, the step of making available a contour editing tool of the shape of the contour.

3. Software according to claim 1, wherein:
the step of identifying a contour, comprises identifying a respective contour for each anatomical part illustrated in the two-dimensional image,
the step of generating a two-dimensional image, comprises generating a two-dimensional image containing the contours of the identified anatomical parts,
the step of allowing the selection of a medical characteristic, comprises allowing the selection of another medical characteristic,
the step of allowing the association, comprises allowing the association of the other medical characteristic to another contour of the plurality of contours present in the two-dimensional image of the contours of the anatomical parts,
the step of filling the two-dimensional image comprises filling the two-dimensional image of the contours of the anatomical parts with the colour of the other respective selected medical characteristic, filling with the colour only the portion of said two-dimensional image which is within the other contour to which the other medical characteristic has been associated,
in the step of generating a report, wherein that the report contains the two-dimensional image of the contours previously filled with the colour and the character strings associated with the selected medical characteristics.

4. Software according to claim 1, comprising, subsequent to the step of displaying said two-dimensional image of the contour, the step of making a contour deletion tool available.
